(19) United States
(12) Patent Application Publication
(10) Pub. No.: US 2026/0234117 A1
(43) Pub. Date: Aug. 13, 2026

(54) PROCESS FOR THE PREPARATION OF 5-(ALKOXYMETHYL)FURFURAL

(71) Applicant: Avantium Knowledge Centre B.V., Amsterdam (NL)

(72) Inventors: Gerardus Johannes Maria Gruter, Amsterdam (NL); Gerardus Petrus Maria Van Klink, Amsterdam (NL); Jorge Bueno Moron, Amsterdam (NL)

(21) Appl. No.: 19/157,919

(22) PCT Filed: Feb. 19, 2024
(86) PCT No.: PCT/EP2024/054109

§ 371 Date: Aug. 19, 2025

(30) Foreign Application Priority Data
Feb. 20, 2023 (EP) 23157610.9

(51) Int. Cl. C07D 307/50

(52) U.S. Cl. CPC C07D 307/50

(57) ABSTRACT

A process comprising (i) contacting 5-(chloromethyl)furfural with an alcohol selected from the group consisting of methanol and ethanol, to obtain a product mixture containing 5-(alkoxymethyl)furfural, alcohol and hydrochloric acid, (ii) separating a side stream comprising at least part of the alcohol and hydrochloric acid from the product mixture to obtain 5-(alkoxymethyl)furfural product, (iii) adding water to the side stream to obtain an aqueous mixture, and (iv) separating the aqueous mixture into an aqueous hydrochloric acid and alcohol.

### FIELD OF THE INVENTION

The present invention relates to a process for converting 5-(chloromethyl)furfural (CMF) to produce 5-(alkoxymethyl)furfural.

### BACKGROUND TO THE INVENTION

In recent times use of sustainable resources, such as biomass, is becoming increasingly important for the production of compounds for fuel and chemical applications. Such bio-derived fuels and chemicals are sometimes also referred to as “biofuels” and “biochemicals”. One of the advantages of using sustainable biomass resources is that the CO₂ balance is more favorable as compared with a conventional feedstock of a mineral source. The production of biofuels and biochemicals from a non-edible sustainable resource, such as solid lignocellulosic material, is preferred, as such non-edible solid lignocellulosic material does not compete with food production. Lignocellulosic material can be converted into CMF which subsequently can be converted further.

WO2019149843 describes a two-step staged hydrolysis of lignocellulosic biomass followed by only taking the hydrolysed cellulosic saccharides to the CMF production step for the production of subsequent CMF derivatives. WO2019149853 describes a one-step hydrolysis of lignocellulosic biomass followed by taking the hydrolysed hemicellulosic and cellulosic saccharides to the CMF production step for the production of subsequent CMF derivatives. Although the conversion of 5-(chloromethyl)furfural into 5-(alkoxymethyl)furfural has been described in WO2019149853 and WO2019149843, these documents are relatively silent on the downstream processing of the reactor effluent.

5-(Alkoxymethyl)furfural compounds are interesting both for fuel (additives) and chemical applications. For example, 5-(ethoxymethyl)furfural (EMF) is an interesting diesel fuel additive. Further, for example 5-(methoxymethyl)furfural (MMF) is an important intermediate in the production of 2,5-furandicarboxylic acid (FDCA). Estonian patent application EE2013/0003A describes a method for the preparation of 5-(alkoxymethyl)furfurals from 5-(chloromethyl)furfural or 5-(bromomethyl)furfural.

It is desirable to have a process that would allow to produce 5-(alkoxymethyl)furfural especially by starting from lignocellulose. The production of some side products such as furfural, also can be economically interesting. This would be especially attractive if the production of such side-products would be flexible for example depending on market demand. Such flexibility can also allow one to ensure that the highest efficiency can be obtained based on the kind and amount of wood available.

When converting 5-(chloromethyl)furfural into 5-(methoxymethyl)furfural or into 5-(ethoxymethyl)furfural using excess methanol or ethanol, an effluent results that contains the target product 5-(methoxymethyl)furfural or 5-(ethoxymethyl)furfural, an equivalent amount of HCI and the excess methanol or ethanol besides any side products. As described for example for EMF in the supplementary material of the paper Mascal, M. et al. Angew Chem Int Ed 2008, 47, 7924: “Direct, High-Yield Conversion of Cellulose into Biofuel”, at least methanol or ethanol plus HCI are to be separated from the 5-(methoxymethyl)furfural or 5-(ethoxymethyl)furfural comprising product in order for the process to be economically viable. Additionally, It would be desirable to separate, if present, any residual extraction solvent that was used in the production and isolation of 5-(chloromethyl)furfural.

### SUMMARY OF THE INVENTION

The present invention now relates to a process comprising (i) contacting 5-(chloromethyl)furfural with an alcohol selected from the group consisting of methanol and ethanol, to obtain a product mixture containing 5-(alkoxymethyl)furfural, alcohol and hydrochloric acid, (ii) separating a side stream comprising at least part of the alcohol and hydrochloric acid from the product mixture to obtain 5-(alkoxymethyl)furfural product, (iii) adding water to the side stream to obtain an aqueous mixture, and (iv) separating the aqueous mixture into an aqueous hydrochloric acid and alcohol.

The side stream obtained in step (ii) also can be referred to as a reagent stream. Hereinafter we will refer to it as a side stream.

The expression “5-(alkoxymethyl)furfural” hereinafter in the description means “5-(methoxymethyl)furfural and/or 5-(ethoxymethyl)furfural”. The expression “alcohol” hereinafter in the description means “methanol and/or ethanol”.

The hydrochloric acid in the side stream mixture makes that this mixture as such is not attractive for further use. Surprisingly, the present process allows to separate from 5-(alkoxymethyl)furfural a stream comprising alcohol and hydrochloric acid in a form which is suitable for further use.

In the present process, a side stream is separated from the product mixture containing 5-(alkoxymethyl)furfural after which water is added to this side stream. Such subsequent addition of water makes it easy to separate alcohol from the resultant aqueous hydrochloric acid. The resultant aqueous hydrochloric acid solution will contain water and hydrochloric acid and can further contain alcohol. The separation conditions tend to be chosen such that the alcohol which is separated off mainly consists of the alcohol, generally consists for at least 95% by weight of alcohol.

### DETAILED DISCUSSION

The 5-(chloromethyl)furfural for use in the present process can be prepared in any way known to the person skilled in the art. Preferably, the 5-(chloromethyl)furfural is obtained from lignocellulose. Many processes have been described for converting lignocellulose into 5-(chloromethyl)furfural. The process as described in the article by Mascal et al. titled “Dramatic Advancements in the Saccharide to 5-(chloromethyl)furfural Conversion Reaction”, published in ChemSusChem (2009), vol 2, pages 859-861 has the disadvantage that it lacks flexibility in respect of the type of solid lignocellulosic material used as a feedstock. Furthermore, all processes according to this article treat the lignocellulosic material at a high temperature in the presence of concentrated hydrochloric acid. This tends to lead to substantial production of undesirable side products. A further disadvantage is that some of the side products can have a similar solubility as 5-(chloromethyl)furfural in the solvent such as 1,2-dichloroethane thereby further complicating the isolation and purification of the 5-(chloromethyl)furfural and further decreasing the economical attractiveness of the process.

Advantageous processes for preparing 5-(chloromethyl)furfural are described in WO2019149853 and WO2019149843 as discussed above.

The process of WO2019149843 includes the following steps: a) converting a solid material containing hemicellulose, cellulose and lignin, by: (i) hydrolyzing, at a temperature equal to or less than 40° C., preferably equal to or less than 30° C., at least part of the hemicellulose of the solid material with a first aqueous hydrochloric acid solution, which first aqueous hydrochloric acid solution has a hydrochloric acid concentration in the range from equal to or more than 15.0 wt % to less than 40.0 wt %, based on the weight amount of water and hydrochloric acid in such first aqueous hydrochloric acid solution, yielding a remaining solid material and a hydrochloric acid-containing, aqueous, first hydrolysate product solution; (ii) hydrolyzing, at a temperature equal to or less than 40° C., preferably equal to or less than 30° C., at least part of the cellulose of the remaining solid material with a second aqueous hydrochloric acid solution, which second aqueous hydrochloric acid solution has a hydrochloric acid concentration in the range from equal to or more than 40.0 wt % to equal to or less than 51.0 wt %, based on the weight amount of water and hydrochloric acid in such second aqueous hydrochloric acid solution, yielding a residue and a hydrochloric acid-containing, aqueous, second hydrolysate product solution (b) forwarding to step (c) a, hydrochloric acid-containing, aqueous intermediate product solution comprising: a part of or the whole of the hydrochloric acid-containing, aqueous first hydrolysate product solution of step (a); and/or a part of or the whole of the, hydrochloric acid containing, aqueous second hydrolysate product solution of step (a); and (c) heating at least part of the hydrochloric acid-containing, aqueous intermediate product solution to a temperature equal to or more than 60° C., yielding a product solution containing 5-(chloromethyl)furfural, and extracting the 5-(chloromethyl)furfural from such product solution into an extraction solvent. Further details and preferred features are described in the publication WO2019149843.

The process of WO2019149853 comprises conversion of a solid lignocellulosic material containing hemicellulose, cellulose and lignin, the process including the following steps: a) hydrolyzing, at a temperature equal to or less than 40° C., preferably equal to or less than 30° C., at least part of the hemicellulose and at least part of the cellulose of the solid lignocellulosic material with an aqueous hydrochloric acid solution, containing in the range from equal to or more than 40.0 wt % to equal to or less than 51.0 wt % hydrochloric acid, based on the combined weight amount of water and hydrochloric acid in such aqueous hydrochloric acid solution; yielding a hydrochloric acid-containing, aqueous hydrolysate solution; (b) separating the hydrochloric acid-containing, aqueous hydrolysate solution from the lignin; and (c) heating at least part of the hydrochloric acid-containing, aqueous hydrolysate solution to a temperature equal to or more than 60° C., yielding a product solution containing 5-(chloromethyl)furfural, and extracting the 5-(chloromethyl)furfural from such product solution into an extraction solvent. Further details and preferred features are described in the publication WO2019149853.

The feed for the present process generally will contain other compounds besides the CMF. Therefore, process step (i) preferably is contacting a feed comprising 5-(chloromethyl)furfural with an alcohol selected from the group consisting of methanol and ethanol, to obtain a product mixture containing 5-(alkoxymethyl)furfural, alcohol and hydrochloric acid. Such other compounds present in the feed can be side-products of lignocellulose and further reactants used and/or obtained in the lignocellulose conversion process including but not limited to furfural. Additionally, the feed for the present process can contain extraction solvent which was used for removing CMF from the CMF production process step.

Extraction solvent for use with CMF is preferably an organic extraction solvent. The extraction solvent can for example be a non-polar solvent or an aprotic polar solvent. Preferably the extraction solvent is an organic extraction solvent, more preferably an organic extraction solvent selected from the group consisting of: C6-C10 aromatic hydrocarbons, C1-C10 chlorinated hydrocarbons and C3-C10 ketones and mixtures of two or more thereof. By a Cx compound is herein understood a compound comprising “x” carbon atoms. By a Cx-Cz compound is herein understood a compound comprising in the range from “x” to “z” carbon atoms. Suitably the extraction solvent can be selected from the group consisting of: diethyl ether, diisopropyl ether, ethyl acetate, pentane, hexane, heptane, octane, decane, dodecane, cyclohexane, benzene, toluene, xylene, dichloromethane, dichloroethane, carbon tetrachloride, trichloromethane (chloroform), methyl tert-butyl ether, and mixtures of two or more thereof. Most preferably an aromatic extraction solvent is used, more preferably selected from the group consisting of benzene, toluene and xylene. More preferably, the extraction solvent is therefore selected from the group consisting of heptane, octane, decane, dodecane, toluene, xylene, 1,2-dichloroethane, carbon tetrachloride and mixtures of two or more thereof. Preferably, the extraction solvent is toluene or 1,2-dichloroethane.

A further group of preferred solvents are halogenated aromatic extraction solvents more especially one or more compounds selected from the group consisting of mono-chlorobenzene, o-dichlorobenzene, m-dichlorobenzene, p-dichlorobenzene, 1,2,4-trichlorobenzene, mono-fluorobenzene, o-difluorobenzene, m-difluorobenzene, p-diflurorobenzene, 1,2,4-trifluorobenzene, 1,3-dichloro-2-fluorobenzene, fluorotoluene and chlorotoluene. Most preferred is fluorobenzene.

Preferably, the 5-(chloromethyl)furfural is processed in the present process in a non-isolated form so without being separated from the extraction solvent. That is, preferably the mixture containing 5-(chloromethyl)furfural and extraction solvent is further processed as a whole.

Alternatively, 5-(chloromethyl)furfural is first isolated from the extraction solvent, before converting such 5-(chloromethyl)furfural in the present process. In such case, the 5-(chloromethyl)furfural can be isolated from the extraction solvent in any manner known by the person skilled in the art, for example by evaporation, distillation and/or crystallization. After removal of the 5-(chloromethyl)furfural, the extraction solvent may be recycled for reuse.

The 5-(chloromethyl)furfural is reacted with an alcohol selected from the group consisting of methanol and ethanol. It is preferred to have a molar excess of alcohol present to enable to speed up the reaction. The molar ratio of alcohol to 5-(chloromethyl)furfural in step (i) can be of from 30 to 1, more specifically of from 20 to 1, more specifically of from 10 to 1.5:1, more specifically of from 10 to 3, all molar equivalent of alcohol relative to the molar amount of 5-(chloromethyl)furfural.

The 5-(chloromethyl)furfural is contacted and reacted with alcohol preferably at a temperature of from room temperature to the ambient pressure boiling point of the mixture thereby producing 5-(methoxymethyl)furfural or 5-(ethoxymethyl)furfural and hydrochloric acid co-product. Preferably, the temperature applied is up to 80° C., preferably of from 30 to 80° C., more specifically of from 30 to 50° C. If the intention is to reduce the amount of acetal produced and thereby improve the yield, the temperature is at most 50° C., more specifically at most 40° C., more specifically at most 35° C., most specifically ambient temperature. As will be clear to the person skilled in the art, the pressure to be applied will depend on the compounds present.

The side stream is separated in step (ii) from the mixture obtained in step (i). The separation generally will be carried out at a temperature of from 0 tot 60° C. Generally, it is preferred to apply from ambient temperature to 60° C.

The side stream will contain at least part of the alcohol and at least part of the hydrochloric acid present in the product mixtures obtained in step (i). The side stream can contain unreacted alcohol and alcohol obtained from conversion of acetals into the corresponding 5-(chloromethyl)furfural and 5-(alkoxymethyl)furfural.

The 5-(alkoxymethyl)furfural preferably is in contact with hydrochloric acid at elevated temperature during limited time to prevent further conversion into undesired side-products. Therefore, it is preferred to lower the temperature and/or substantially remove the hydrochloric acid from the product mixture shortly after the desired conversion has been attained.

The side stream comprising methanol and HCI, or ethanol and HCI, can be removed from the 5-(alkoxymethyl)furfural product. Preferably, the side stream is removed in the form of a gas or vapor from the 5-(alkoxymethyl)furfural product which tends to be liquid. Suitable separation methods are evaporation and distillation. The residual higher boiling fraction, containing 5-(alkoxymethyl)furfural optionally with residual extraction solvent, can be withdrawn from the reactor and processed further. High boiling components are components having boiling points at ambient pressure of higher than 100° C.

Alternatively, the side stream is separated in step (ii) from the reaction mixture of step (i) with the help of a membrane.

In step (iii) water is added to the side stream comprising hydrogen chloride and alcohol. The amount of water tends to be of from 100 to 1 molar equivalent of water relative to the hydrogen chloride present, more specifically of from 50 to 1, more specifically of from 20 to 1, more specifically of from 8 to 1.

In step (iv), the aqueous mixture obtained is separated into aqueous hydrochloric acid and alcohol. The separation can be in any way known to the person skilled in the art including but not limited to evaporation and distillation of the alcohol from the aqueous HCI solution. This evaporation preferably is performed such that the methanol and ethanol that is obtained overhead is substantially free from HCI, where substantially free means preferably less than 1 mol % of HCI relative to the molar amount of alcohol. At the same time, the residual aqueous HCI solution is substantially free of methanol and ethanol, substantially free meaning less than 0.1 mol methanol or ethanol per liter aqueous HCI.

Thus with a process according to the invention, the downstream separation of the reaction mixture makes it possible to recycle the methanol or ethanol and the HCI back into the CMF conversion step and into the CMF production step, respectively.

The alkoxymethylfurfural so obtained is suitable for preparation of 2,5-furan-dicarboxylic acid (FDCA). Therefore, a preferred process further comprises (a) oxidizing 5-(alkoxymethyl)furfural product using an oxidizing gas at elevated temperature in the presence of a catalyst system; (b) separating from the reaction mixture obtained in step (a) an overhead stream and crude carboxylic acid composition comprising 2,5-furandicarboxylic acid; and (c) further separating the crude carboxylic acid composition comprising 2,5-furandicarboxylic acid in a separation zone to obtain solid containing 2,5-furandicarboxylic acid and effluent. The crude product can be purified by (d) washing the solid containing 2,5-furandicarboxylic acid with washing solution comprising acetic acid. The purity of the product can be further improved by (e) contacting washed solid containing 2,5-furandicarboxylic acid with polar solvent to obtain a solution; (f) contacting the solution with hydrogen in the presence of a hydrogenation catalyst at hydrogenation conditions yielding a hydrogenated solution; and (h) separating purified 2,5-furandicarboxylic acid from the hydrogenated solution.

Hereinafter, the invention is described in more detail using experiments.

### Example 1

The experiments were performed in small batch reactor tubes (9 mL volume) which were equipped with magnetic stirrers. These were heated in custom made alumina heating blocks.

The reactor tubes were loaded with the desired amount of CMF to which MeOH was added. The reactor tubes were closed and placed inside the alumina block and were allowed to react while mixing for the time period indicated at room temperature. The product was analyzed by Gas Chromatography for the content of residual CMF, MMF, the corresponding acetals and decomposition products without further work-up. Only the desired compounds are reported.

TABLE 1
Reactor loadings of CMF and MeOH

| | | | MeOH |
|---|---|---|---|
| Sample | CMF | MeOH | equivalents |
| nr | (mmol) | (mL) | vs. CMF |
| 1 | 0.248 | 3 | 300 |
| 2 | 0.691 | 3 | 107 |
| 3 | 1.418 | 3 | 52 |
| 4 | 0.440 | 0.45 | 25 |
| 5 | 0.762 | 0.42 | 14 |
| 6 | 0.726 | 0.25 | 8.5 |
| 7 | 1.453 | 0.34 | 5.8 |

TABLE 2
Molar yields of MMF (acetals)

| | | Yield (molar %, based on CMF feed) | | | | | | | | | |
|---|---|---|---|---|---|---|---|---|---|---|---|
| | | 1 h | | | 4.5 h | | | 22 h | | | |
| Sample | CMF | | MMF | Total | | MMF | Total | | MMF | Total | HCl after |
| nr | (wt %) | MMF | acetal | product | MMF | acetal | product | MMF | acetal | product | 22 h (wt %) |
| 1 | 1.5 | 15.5 | 51.6 | 67.1 | 31.1 | 64.9 | 96.0 | 42.0 | 54.2 | 96.2 | 0.4 |
| 2 | 4.2 | 31.3 | 39.4 | 70.7 | 51.9 | 44.9 | 96.8 | 61.3 | 33.6 | 94.9 | 1.1 |
| 3 | 8.6 | 49.1 | 15.2 | 64.3 | 73.8 | 17.8 | 91.6 | 77.2 | 14.6 | 91.8 | 2.3 |
| 4 | 17.85 | 36.9 | 19.6 | 56.5 | 57.0 | 18.6 | 75.6 | 46.7 | 29.8 | 76.5 | 5.1 |
| 5 | 33.13 | 28.5 | 19.0 | 47.5 | 49.0 | 15.5 | 64.5 | 29.3 | 16.2 | 45.5 | 10.7 |
| 6 | 52.98 | 30.1 | 10.8 | 40.9 | 39.1 | 21.9 | 61.0 | 22.5 | 6.2 | 28.7 | 20.6 |
| 7 | 77.99 | 24.1 | 5.3 | 29.4 | 37.0 | 7.1 | 44.1 | 10.7 | 3.9 | 14.6 | 40.8 |

### Example 2

To 1.185 g of a 3 M HCI in MeOH solution (density 0.876, corresponding to 4.0 mmol HCI) was added 0.68 g of water. Subsequently, the MeOH was evaporated overnight by a gentle nitrogen flow at atmospheric pressure and room temperature. The remaining liquid (0.750 g) was titrated with NaOH and found to contain 16.44 wt % HCI. Thus, 90% of HCI was recovered.

1. A process comprising:
(i) contacting 5-(chloromethyl)furfural with alcohol selected from the group consisting of methanol and ethanol, to obtain a product mixture containing 5-(methoxymethyl)furfural and/or 5-(ethoxymethyl)furfural, alcohol and hydrochloric acid,
(ii) separating a side stream comprising at least part of the alcohol and hydrochloric acid from the product mixture to obtain 5-(methoxymethyl)furfural and/or 5-(ethoxymethyl)furfural product,
(iii) adding water to the side stream comprising at least part of the alcohol and hydrochloric acid, to obtain an aqueous mixture comprising alcohol and hydrochloric acid, and
(iv) separating the aqueous mixture comprising alcohol and hydrochloric acid into an aqueous hydrochloric acid and alcohol.

2. The process according to claim 1, wherein the side stream is removed from the product mixture in the form of gas or vapour.

3. The process according to claim 1, wherein the side stream is separated off at a temperature of up to the boiling point of the mixture present in step (i).

4. The process according to claim 1, wherein the alcohol separated off in step (iv) is sent back to step (i).

5. The process according to claim 1, wherein the 5-(chloromethyl)furfural is obtained by conversion of lignocellulose.

6. The process according to claim 1, wherein the alcohol is separated in step (iv) from the aqueous mixture in the form of a gas or vapour.

7. The process according to claim 1, wherein the side stream is removed from the product stream with the help of a membrane.

8. The process according to claim 1, which process further comprises;
a) oxidizing 5-(alkoxymethyl)furfural product using an oxidizing gas at elevated temperature in the presence of a catalyst system;
b) separating from the reaction mixture obtained in step a) an overhead stream and crude carboxylic acid composition comprising 2,5-furandicarboxylic acid; and
c) further separating the crude carboxylic acid composition comprising 2,5-furandicarboxylic acid in a separation zone to obtain solid containing 2,5-furandicarboxylic acid and effluent.

9. The process according to claim 8, which process further comprises:
d) washing the solid containing 2,5-furandicarboxylic acid with washing solution comprising acetic acid.

10. The process according to claim 9, which process further comprises;
e) contacting washed solid containing 2,5-furandicarboxylic acid with polar solvent to obtain a solution;
f) contacting the solution with hydrogen in the presence of a hydrogenation catalyst at hydrogenation conditions yielding a hydrogenated solution; and
g) separating purified 2,5-furandicarboxylic acid from the hydrogenated solution.
